(19) United States
(12) Patent Application Publication
(10) Pub. No.: US 2007/0076401 A1
(43) Pub. Date: Apr. 5, 2007

(54) Male connectors and female connectors which are used to produce liquid transmission connections, such as for enteral nutrition lines

(75) Inventors: Jean-Luc Carrez, Ecouen (FR); Valery Dalle, Gouvieux (FR); Pierrick Guyomarc's, Ermont (FR); Michel Tempereau, Luzarches (FR)

Correspondence Address: LEVINE & MANDELBAUM, 444 MADISON AVENUE, 35TH FLOOR, NEW YORK, NY, 10022, US

(73) Assignee: Vygon, Ecouen (FR)

(21) Appl. No.: 10/596,009

(22) PCT Filed: Dec. 3, 2004
(86) PCT No.: PCT/FR04/03114

§ 371 Date: May 25, 2006

(30) Foreign Application Priority Data
Dec. 5, 2003 (FR) 0314292

(51) Int. Cl. H05K 9/00

(52) U.S. Cl. 361/816

(57) ABSTRACT

The invention relates to connectors which are intended for use in the medical field. The entry diameter and the thread crest diameter of the inventive connectors are selected in relation to the corresponding diameters of standard connectors such that the assembly of a male connector (A, C) or a female connector (B, D) respectively with a standard female or male connector is prevented because the end piece of the male connector cannot penetrate the inlet duct of the female connector or because said penetration is stopped when the head of the female connector comes into contact with the flange ring of the male connector. The invention is suitable, for example, for enteral nutrition lines.

The invention concerns connectors for conical assembly and with locking thread, which can be used to create liquid transmission connections in the area of medical coupling, and in particular for enteral nutrition lines.

An enteral nutrition line generally includes a nutrient container connected by a flexible tube to an enteral nutrition probe.

These three components must be connected step by step.

Normally, the container has a connecting end which constitutes, or which is equipped with, a male connector, and the nutrition probe has a connecting end which constitutes or which is equipped with a female connector.

The male connector of the container can be connected directly to the female connector of the probe, but most often this connection is achieved by means of a flexible tube which is terminated at one end with a female connector capable of connecting to the male connector of the container, and which is terminated at its opposite end with a male connector capable of connecting to the female connector of the probe.

Connectors are also used in other medical devices, such as in perfusion devices for intravenous or arterial catheters, etc.

Standards have been established for all of these connectors, imposing certain dimensions upon them.

Standard NF IN 20 594 concerns connectors designed to create conical assemblies with an imposed taper of 6% (the Luer standard), and in particular determining the minimum and maximum entry diameters of the connectors.

According to standard NF IN 20 594, the male connectors for Luer conical assemblies have an entry diameter that falls within the range 3.925 mm to 3.990 mm (for rigid material) or within the range 3.925 mm to 4.027 mm (for semi-rigid material), and the female connectors have an entry diameter that falls within the range 4.270 mm to 4.315 mm.

The entry diameters are the diameters which determine the ability to fit the male connector inside the female connector. It therefore concerns the outside diameter in the case of the male connector, and the inside diameter in the case of the female connector.

Standard NF IN 1707 describes connectors for conical assembly that are equipped with locking threads capable of fitting together to achieve locking of the connection, and in particular specifies the diameters at the base of the threads and at the crest of the threads of these connectors.

According to standard NF 1 707, the threads of the Luer conical connectors meet the following conditions:

for the male connector, the diameter of the thread at the base must be 8.00 mm and the diameter of the thread at the crest must be 7.00 mm,

for the female connector, the diameter of the thread at the base must be 6.73 mm maximum and the diameter of the thread at the crest must be 7.83 mm.

The standardised female connectors have a head which determines the entry conduit of the connector, and on the outer face of which is formed the thread, so that the outside diameter of this head is also the diameter of the threads at the crest, while the standardised male connectors have a projecting tube which determines the entry conduit of the male connector, and which is surrounded by a fixed or mobile collar that forms a channel around the tube in order to receive the head of a corresponding female connector, and on the inner face of which is formed the thread of the connector so that the diameter at the crest of the threads of the male connector determines the passage diameter of the connector channel.

For assembly, the head of the male tube is able to enter, with lateral sealing, into the entry conduit of the female connector, the head of the female tube is able to enter into the channel of the male connector, and the thread of the two connectors is capable of fitting together in order to lock the assembly.

The fact that these standards apply without distinction to connectors for enteral nutrition and to connectors for venous perfusion is a potential cause of accidents.

In fact it can happen that the nutrient container, a syringe for example, can be used by accident to feed a perfusion line or a venous or arterial catheter, and it can happen that a container holding a product other than a nutrient may be used to supply a probe that is employed for enteral nutrition.

Various measures have been recommended to avoid these undesirable connections. It has been suggested that the connectors should be distinguished by colour codes, and this has proven to be an inadequate precaution.

In the case of syringes, it has been proposed (in patent FR 787 999) that these be fitted with a Luer female end instead of the normal Luer male end.

People have also proposed (in patent FR 2 801 987) the use of non-Luer male connectors fitted with an end beading.

Publication WO 01/83001 describes medical connectors that have unconventional diameters designed to be capable of being coupled together or to be coupled with conventional connectors by means of adapters.

In fact this publication aims to be able to use connectors that have inside diameters which are greater than those of the conventional connectors, and to be able to connect them nevertheless to conventional connectors if necessary, by means of adapters.

Publication US 3 751 077 describes metal connectors for high pressure or high voltage.

Publication GB 2 383 828 aims to prevent connection errors in the medical area, and to this end it recommends connectors which it describes as “different”, designed so as not to be able to connect to standard connectors.

According to this publication, it is possible to achieve this difference by providing the male connector with a taper other than 6%, so that this connector does not mate with a standard female connector with a Luer taper, that is one with a taper of 6%.

A simple difference of taper does not in itself prevent the insertion of the different male connector into the standard female connector, so that a connection error remains possible even if the connection is not perfect.

Another difference recommended in this publication concerns a reduction in the diameter of the male connector. It is clear that this reduction, though it can prevent perfect connection, does not prevent the insertion of the reduced male connector into the standard female connector—quite the contrary in fact—so that the risk of a connection error still remains.

Finally, publication GB 2 383 828 recommends the creation of a mismatch between the reciprocal threads of the crown of the male connector and the female connector. This mismatch does not mean that the male connector cannot be inserted into the female connector, so that the risk of a bad connection still remains.

In fact publication GB 2 383 828 essentially aims to prevent the locking of a different connector and a standard connector, but the risk remains of a connection that is not actually perfect but that is sufficient to deceive the user.

This present invention aims to prevent all manipulation errors, and not just a locking error.

This is achieved in the invention by using male and female connectors for conical assemblies and with locking threads, which differ from the standardised connectors by the fact that they have an entry diameter and a diameter at the crest of the threads that are chosen in relation to the corresponding diameters of the standardised connectors so that the assembly of a male connector (RMI) or female connector (RFI) according to the invention, using a standardised female connector (RFN) or standardised male connector (RMN) respectively, is prevented because penetration of the tube of the male connector into the entry conduit of the female connector is impossible, or because this penetration is halted by the head of the female connector butting against the collar of the male connector.

For example, the connectors of the invention have the characteristic dimensions indicated in the following table:

| Diameter (mm) | RMI | RFN | RFI | RMN |
|---|---|---|---|---|
| Entry | 3.2 | 4.270-4.315 | 3.5 | 3.925-4.027 |
| At the base of the threads | 7 | 6.73 | 5.6 | 8 |
| At the crest of the threads | 5.8 | 7.83 | 6.8 | 7 |
| Entry | 4.5 | 4.270-4.315 | 4.8 | 3.925-4.027 |
| At the base of the threads | 8.4 | 6.73 | 7.2 | 8 |
| At the crest of the threads | 7.4 | 7.83 | 8.2 | 7 |

The tube of a male connector according to the invention (RMI), with an entry diameter of 3.2 mm and a diameter at the crest of the threads of 5.8 mm, can penetrate into the entry conduit of a standardised female connector (RFN), but this penetration will be halted by the head of the female connector butting against the collar of the male connector, and a female connector according to the invention (RFI), with an entry diameter of 4.8 mm and a diameter at the crest of the threads of 8.2 mm, is allowed to penetrate by the ferrule of a standardised male connector, but this penetration will be halted by the head of the female connector butting against the collar of the male connector.

In addition, the tapered connectors according to the invention preferably have a taper other than the Luer taper, such as a taper of 4% or indeed of 8-10%.

In particular implementations, the connectors of the invention have the following additional characteristics, in combination or not:
a male cone, non Luer, at 8% (instead of 6%), that is with a slope of 2° 17′26″(instead of 1° 43′6″)
a female cone, of the non-Luer type, at 8%
a male or female cone length of 6.5 mm,
locking with double thread screw with a pitch of 5 mm.

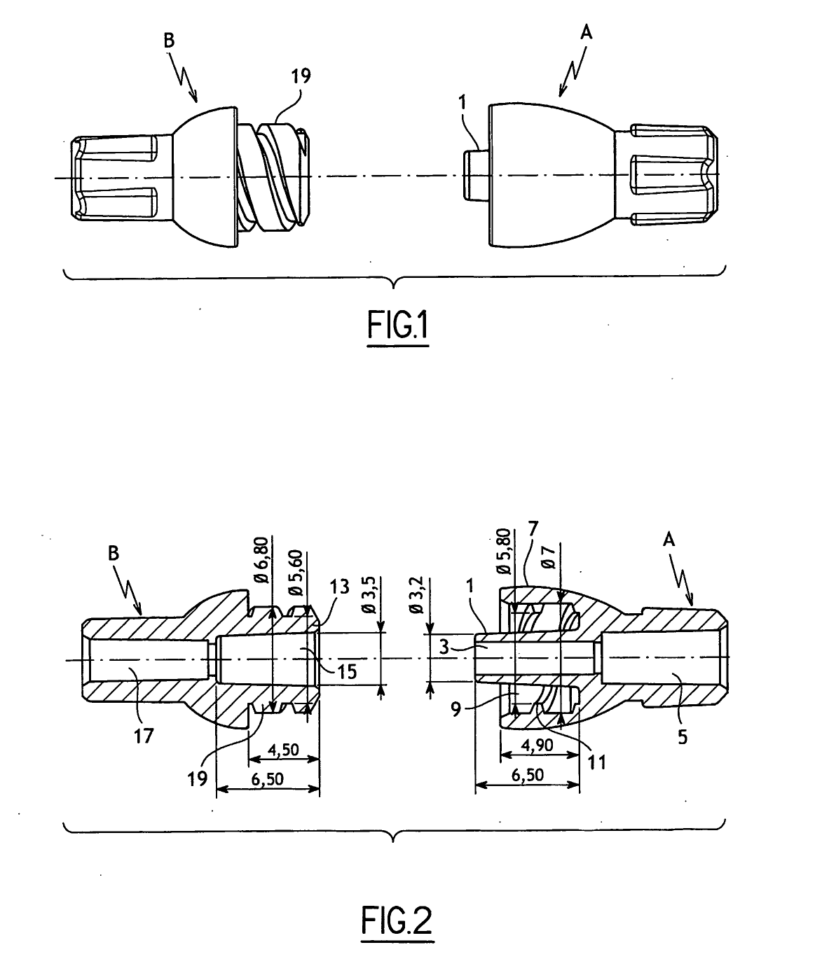

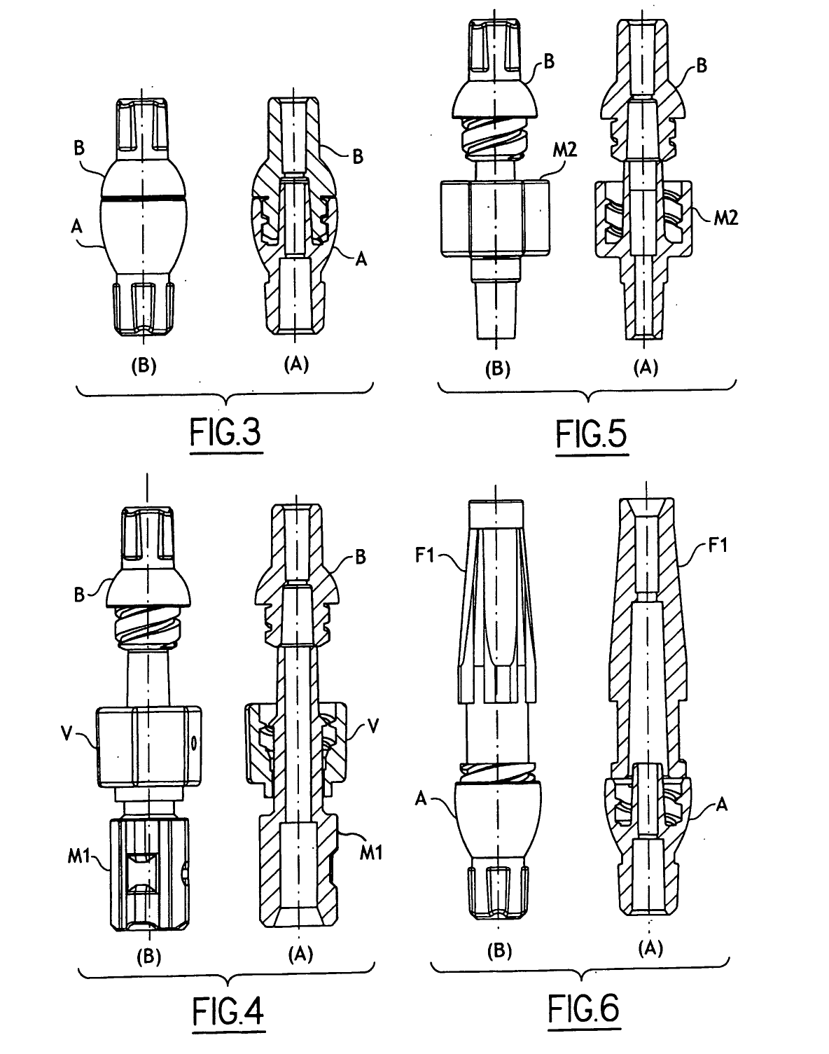

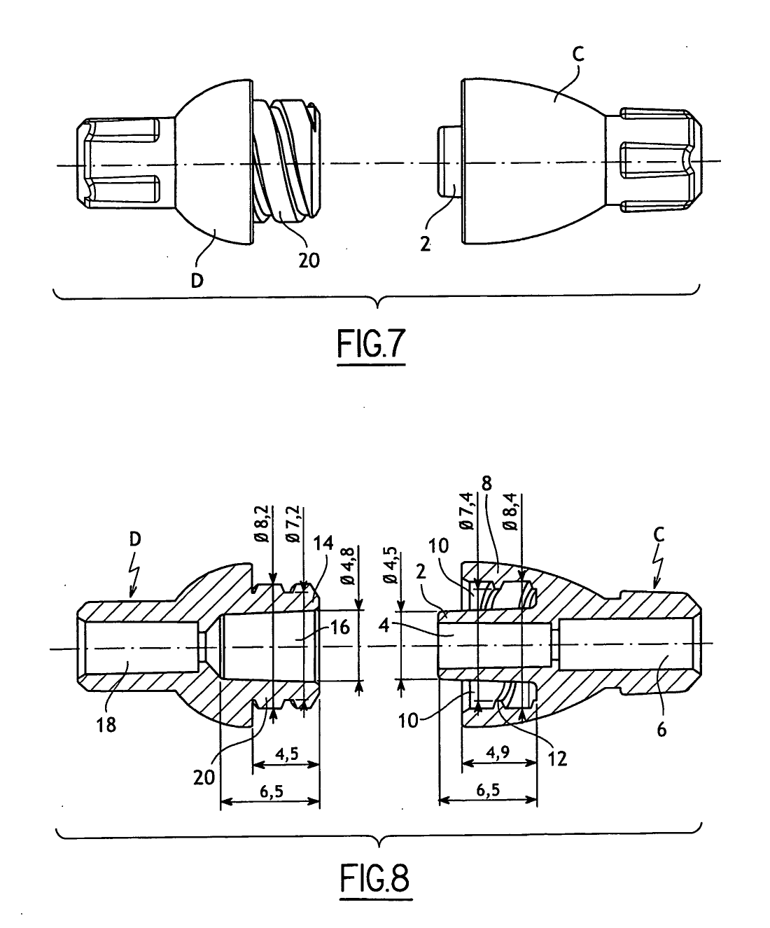

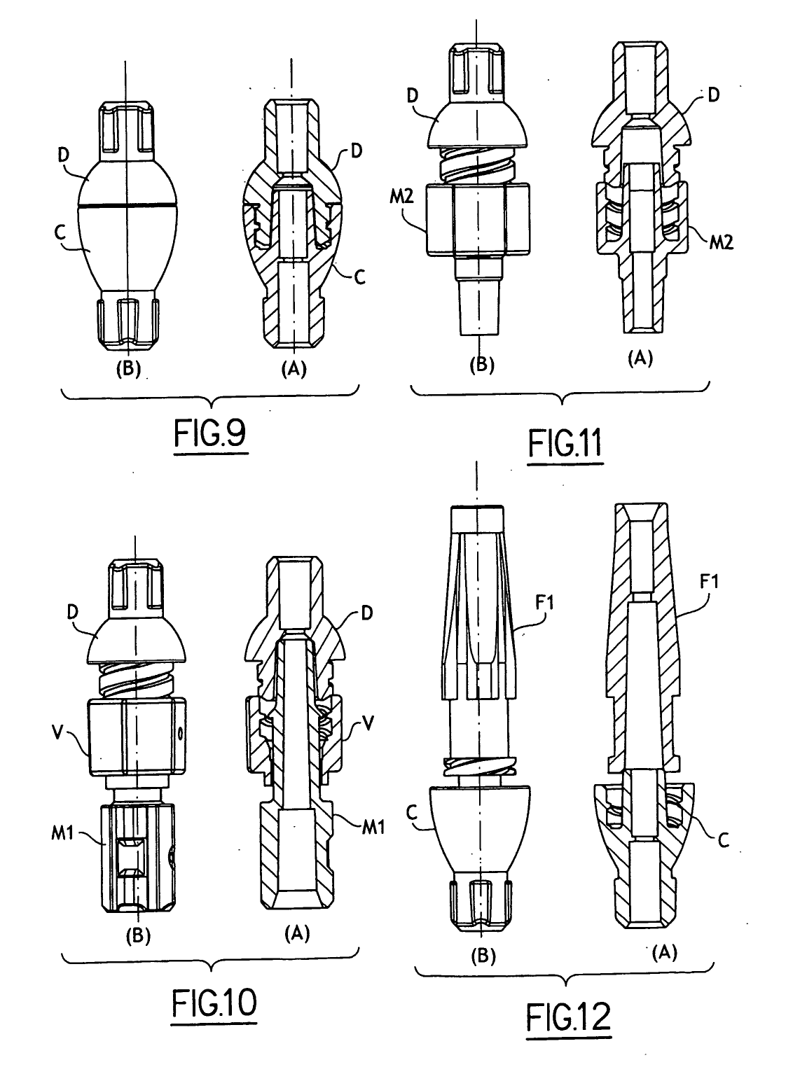

The drawings in the attached figures illustrate examples of a male connector (A or C) and a female connector (B or D) according to the invention and designed to be coupled.
FIG. 1 is an axial section of a male connector and the corresponding female connector, according to the invention,
FIG. 2 is an axial section of the connectors of FIG. 1,
FIG. 3 shows, in axial section (FIG. 3 (A)), and in perspective (FIG. 3 (B)), the assembly of the connectors of FIG. 1,
FIGS. 4 to 6 are views in axial section and the corresponding perspective, illustrating the inability to couple a male or female connector according to FIG. 1 using standardised connectors,
FIG. 7 is a view in perspective of another male connector and the corresponding female connector, according to the invention,
FIG. 8 is an axial section of the connectors of FIG. 7.
FIG. 9 shows, in axial section (FIG. 9 (A)) and in perspective (FIG. 9 (B)), the assembly of the connectors of FIG. 6, and
FIGS. 10 to 12 are axial sections and the corresponding perspectives illustrating the inability to couple a male or female connector according to FIG. 6 using standardised connectors.

In a manner which is known as such, the male connectors, A and C of the invention have a projecting end tube (1 ; 2) which forms a conical entry passage (3 ; 4) communicating with a rear channel (5 ; 6) and having, around the tube, a collar (7 ; 8) which forms a channel (9 ; 10) around the tube, and the collar has an internal thread (11 ; 12) oriented toward the tube.

In a manner which is known as such, the female connectors (B and D) of the invention have a front head (13 ; 14) which forms a conical entry conduit (15 ; 16) that communicates with a rear channel (17 ; 18), and this head is provided with an external thread (19 ; 20).

The characteristic dimensions of the connectors are shown in FIGS. 1 and 7.

The head of the female tube B is able to enter into the channel of the male tube A and to be screwed onto it, while the head of the female tube D is able to enter into the channel of the male tube C and to be screwed onto it.

The male tube A is able to enter, with lateral sealing, into the entry conduit of the female connector (B), and the male tube C is able to enter, with lateral sealing, into the entry conduit of the female connector D.

The rear channels of the male connectors and of the female connectors are used to attach the connectors to tubes, in a manner which is known as such.

The dimensions of the male connectors A and C and of the female connectors B and D are chosen so as to allow the assembly of connectors A and B as shown in FIG. 3, and the assembly of connectors C and D as shown in FIG. 9.

On the other hand, these connectors cannot be assembled with standardised connectors, as shown in FIGS. 4 to 6 and 10 to 12 for example.
In FIG. 4, the tube of the standardised male connector M1, with sliding locking latch V, is unable enter into the entry conduit of the female connector B of the invention.
In FIG. 5, the tube of the standardised male connector M2, with fixed collar, is unable to enter into the entry conduit of the female connector B of the invention.
In FIG. 6, the tube of the male connector A of the invention can enter into the entry conduit of the standardised female connector F1, but the head of the female connector is unable to enter into the channel of the male connector,
In FIG. 10, the tube of the standardised male connector M1, with sliding locking latch V, can enter into the entry conduit of the female connector D according to the invention, but the head of this female connector is unable to enter into the channel of the latch.
In FIG. 11, the tube of the standardised male connector M2 can enter into the entry conduit of the female connector D of the invention, but the head of this female connector is unable to enter into the channel of the male connector.
In FIG. 12, the tube of the male connector (C) of the invention is unable to enter into the entry conduit of the standardised female connector F1.

These examples of incompatibility are not exhaustive.

The male connectors (RMI) and female connectors (RFI) of the invention are designed in particular to be fitted to miscellaneous containers (syringes, syringes fitted with a pipette, force-feeders, sachets, flasks, or bottles), probes, tubes, or three-way connectors.

As a result, the invention also includes:
a container (in particular a sachet, flask, bottle, syringe, or force-feeder) fitted with a female connector
a syringe equipped with a pipette in order to take up enteral nutrition products, fitted with a male connector (RMI),
a probe which has a connecting end composed of or equipped with a female connector (RFI),
a tube which has an end equipped with a male connector (RMI),
a connector which has an end composed of a female connector (RFI) and an opposite end which is capable of connecting to an enteral feed container,
a three-way connector, two channels of which are equipped with a male connector (RMI) and a female connector (RFI) respectively, with the third channel being fitted with a male connector (RMI) or a female connector (RFI),
assemblies of connectors that include male connectors (RMI), female connectors (RFI) and standardised connectors, as specified in the table.

The invention is not limited to the methods of implementation described above.

1. A male connector (RMI) and a female connector (RFI) to be assembled and to be locked together in order to create a liquid transmission connection in the area of medical coupling, in which standardised male connectors (RMN) and female connectors (RFN) are currently used, in particular for an enteral nutrition line, with the female connector (RFI) having as the standardised female connector (RFN) a head (13 ; 14) that forms a conical entry conduit (15 ; 16) and which has an external thread (19 ; 20), and with the male connector (RMI) having as standardised male connector (RMN) a projecting conical tube (1 ; 2) that forms an entry conduit (3 ; 4), surrounded by a collar (7 ; 8) which forms a channel (9 ; 10) around the tube, and which has an internal thread (11 ; 12), with the head of the female tube (RFI) being capable of penetrating into the channel of the male connector (RMI), with the head of the male tube (RMI) being capable of penetrating, with lateral sealing, into the entry conduit of the female connector (RFI), and with the threads of the two connectors being capable of fitting together so as to lock the assembly, characterised in that these connectors (RMI, RFI) have an entry diameter and a diameter at the crest of the threads that are chosen in relation to the corresponding diameters of the standardised connectors (RMN, RFN), so that the assembly of a male connector (RMI) or female connector (RFI) with a standardised female connector (RFN) or standardised male connector (RMN) respectively, is prevented because penetration of the ferrule of the male connector into the entry conduit of the female connector is impossible or because this penetration is halted by the head of the female connector butting against the collar of the male connector.

2. Connectors (RMI, RFI) according to claim 1, in which the entry conduits (15 ; 16 - 3 ; 4) have a taper other than 6% (the Luer taper).

3. Connectors (RMI, RFI) according to claim 2, in which the entry conduits (15 ; 16 - 3 ; 4) have a taper falling within the range 4% and 8 - 10%.

4. Connectors (RMI, RFI) according to one of claims 1 to 3, in which the conical entry conduits (15 ; 16 - 3 ; 4) have a length of 6.5 mm.

5. Connectors (RMI, RFI) according to one of claims 1 to 4, in which the threads are double threads with a pitch of 5 mm.

6. Connectors (RMI, RFI) according to one of claims 1 to 5, in which the male connector (A) has an entry diameter of 3.2 mm and a diameter at the crest of the threads of 5.8 mm and in which the female connector (B) has an entry diameter of 3.5 mm and a diameter at the crest of the threads of 6.8 mm.

7. Connectors (RMI, RFI) according to claim 6, in which the male connector (A) has a diameter of 7 mm at the base of the threads.

8. Connectors (RMI, RFI) according to claim 6, in which the female connector (B) has a diameter of 5.6 mm at the base of the threads.

9. Connectors (RMI, RFI) according to one of claims 1 to 5, in which the male connector (C) has an entry diameter of 4.5 mm and a diameter at the crest of the threads of 7.4 mm, and in which the female connector (D) has an entry diameter of 4.8 mm and a diameter at the crest of the threads of 8.2 mm.

10. Connectors (RMI, RFI) according to claim 9, in which the male connector (C) has a diameter of 8.4 mm at the base of the threads.

11. Connectors (RMI, RFI) according to claim 12, in which the female connector (D) has a diameter of 7.2 mm at the base of the threads.

12. Connectors (RMI, RFI) according to one of claims 1 to 11, which have rear channels (5 ; 6, 17 ; 18) that allow attachment of the connectors to tubes.

13. A container fitted with a female connector (RFI) as described in one of claims 1 to 11.

14. A container according to claim 13, from the group composed of a sachet, a flask, a bottle, or a syringe.

15. An enteral nutrition force-feeder, fitted with a female connector (RFI) as described in one of claims 1 to 11.

16. A syringe equipped with a pipette for the take-up of enteral nutrition products, fitted with a male connector (RMI) as described in one of claims 1 to 11.

17. A probe which has a connecting end composed of or equipped with a female connector (RFI) as described in one of claims 1 to 11.

18. A tube which has one end equipped with a male connector (RMI) according to one of claims 1 to 11, and an opposite end equipped with a female connector (RFI) according to one of claims 1 to 11.

19. A connector which has one end composed of a female connector (RFI) according to one of claims 1 to 11, and another end which is capable of connecting to an enteral feed container.

20. A three-way connector in which one channel is fitted with a male connector (RMI) according to one of claims 1 to 11, with each of the other two channels being equipped with a female connector (RFI) according to one of claims 1 to 11.

21. A three-way connector in which one channel is fitted with a female connector (RFI) according to one of claims 1 to 11, with each of the other two channels being equipped with a male connector (RMI) according to one of claims 1 to 11.

22. Enteral nutrition lines fitted with a male connector (RMI) and a female connector (RFI) according to one or more of claims 1 to 11.

23. A set of connectors that include the standardised connectors (RMN, RFN) as specified in the table, and also connectors according to the invention (RMI, RFI) as specified in the table.
